(19) United States
(12) Patent Application Publication
(10) Pub. No.: US 2017/0043067 A1
(43) Pub. Date: Feb. 16, 2017

(54) WOUND CARE DEVICE

(71) Applicant: LOHMANN & RAUSCHER GMBH, SCHOENAU AN DER TRIESTING (AT)

(72) Inventor: JOHANNES SCHNEIDER, WIEN (AT)

(21) Appl. No.: 15/216,857

(22) Filed: Jul. 22, 2016

(30) Foreign Application Priority Data
Jul. 22, 2015 (EP) 15002171.5

(51) Int. Cl. A61M 1/00; A61M 27/00

(52) U.S. Cl. CPC A61M 1/0023; A61M 27/00; A61M 2210/08

(57) ABSTRACT

A wound care device having a covering device fixable to the skin surrounding a wound and used for producing a closed wound space containing the wound. At least sections of the covering device are water vapor-permeable. The covering device preferably comprises a tubular film and a suction port across which a negative pressure can be generated in the wound space. The tubular film preferably has an opening with an opening length of at least 80 cm and more preferably of at least 130 cm.

### CROSS-REFERENCE TO RELATED APPLICATIONS

This application is based on and claims priority to European Patent Application Serial No. 15 002 171.5, filed on Jul. 22, 2015, which is incorporated herein by reference in its entirety.

### STATEMENT REGARDING FEDERALLY SPONSORED RESEARCH OR DEVELOPMENT

Not applicable.

### BACKGROUND OF THE INVENTION

The invention relates to a wound care device having a covering device fixable to the skin surrounding a wound and used for producing an enclosed, at least in portions, water vapor-permeable wound space containing the wound and provided with a tubular film as well as a treatment kit having such a wound care device.

Such wound care devices are used in particular within the framework of the so-called vacuum therapy. It has been found that, in particular, the healing of chronic wounds can be promoted by applying negative pressure to these wounds. In doing so, it has also proven to be advantageous if the wound is covered or filled using open-cell foam or gauze as packing, the wound is covered for creating an enclosed wound space containing the wound and, as the case may be, the packing, and on the side of the covering device facing away from the wound or the packing, a suction port is installed, across which the wound space can be connected to a suction device designed for generating negative pressure. In other arrangements, a flange of the suction port is covered by the covering device or enclosed in a pocket encircling an opening of the covering device. The suction port may, for instance, on the one hand, be provided with a connection device of the suction port, for example designed in the form of a pipe socket and, on the other hand, with a tube that can be connected to a suction device. The covering device may, for instance, be designed as a film-like material, which is hermetically applied to the skin surface adjacent to the wound.

Wound care devices, which are usable within the framework of the vacuum therapy are, for instance, described in EP 0 620 720 B1. With respect to the details of the foaming means and the suction device usable within the framework of the vacuum therapy, the disclosure content of this document is hereby incorporated by express reference into the specification herein. In combination with the characteristics of the main claim, these details may contribute to achieving the technical objective stated hereinafter. Protection is also sought for corresponding combinations of characteristics.

DE 10 2009 019 646 A1 describes a contact layer to be inserted between the packing and the wound base for improving exudate management and forming a drainage space between the packing and the wound base. With respect to the details of the contact layer or the wound covering forming the drainage space, the disclosure content of this document is expressly incorporated into the specification herein. In combination with the characteristics of the main claim, these details may contribute to achieving the technical objective stated hereinafter. Protection is also sought for corresponding combinations of characteristics.

Suction ports, which are usable within the framework of the vacuum therapy and are connectable, across a tube, to a suction device, are, for instance, described in WO 03/073970 A1, WO 2008/014358 A2 and WO 2009/124548 A1. A suction port, called suction head, having projections for flow control in the area of the periphery of the suction port facing the wound, is described in EP 1 018 967 B1. Moreover, a suction port having a contact surface in the form of a disk-like shell to be applied to the packing is specified in EP 1 088 569 B1. For a suction port described in WO 2010/008167 A2, on the periphery facing the packing, channels delimited by webs are formed, through which the wound exudate is to be directed towards an aspiration opening.

WO 2010/011148 A1 describes a wound care device, which is usable within the framework of the vacuum therapy and has an impermeable tube that can be pulled over a limb of the human body as well as a perforated body to be arranged between the wound and the tube. Using the perforated body, a space is created between the impermeable tube and the wound base, in which, across a tube connector that can be applied to the impermeable tube to form a seal, a negative pressure can be generated.

EP 1 162 932 B1 describes a wound care device having an envelope of a plastic material and a fluid-absorbent material contained in the envelope. The wound care device described in this document is intended for protecting wounds. Because of the absence of a tube connector, it is not suitable for use in vacuum therapy.

In EP 2 636 417 A1, a wound care device in accordance with the preamble of claim 1 is provided. The wound care device can have a water vapor-permeable tubular film, which, for treating wounds on the limbs, e.g. foot, ankle, forearm, arm, hand, is pulled over the limb and, with respect to the wound, is positioned in such a way that the wound is covered and sealed by the covering device. Subsequently, using the adhesive film, the covering device can be attached to the skin adjacent to the wound. For this purpose, one may provide for the adhesive film to be pulled off a winder and to be wound around one end of the tube in such a way that, on the one hand, it adheres to the covering device and, on the other hand, to the skin. With respect to the characteristics of adhesive films and to the methods for applying wound care devices, the disclosure content of EP 2 636 417 A1 is thus incorporated into the specification herein by express reference. In combination with the characteristics of the main claim, these details may contribute to achieving the technical objective stated hereinafter. Protection is also sought for corresponding combinations of characteristics.

### BRIEF SUMMARY OF THE INVENTION

The field of application of known wound care devices of the type described above is limited. In view of these prior art problems, the invention is based on the objective of expanding the field of application of wound care devices that are suitable for vacuum therapy. This objective is achieved according to the invention by an enhancement of the prior art wound care devices, which is essentially characterized in that the tubular film has an opening with an opening circumference length of 80 cm or more, especially 130 cm or more.

Such tubular films are usable for wound care, even if the wound is located in the area of a so-called external fixator. It is a retaining system attached through the skin from the outside and is used for immobilizing a part of the body. The invention is based on the idea that a wound care device to be applied in the area of an external fixator can be pulled over the entire external fixator without any detriment to wound healing. Related thereto, it was recognized within the framework of the invention that, other than suggested in EP 2 636 417 and WO 2010/011148 A1, it is not of vital importance to the success of the vacuum therapy to adapt the covering device to the body shape. Instead, an oversized covering device can be readily accepted because this oversize is reduced by applying the negative pressure to the wounds to such an extent that it does not interfere with wound healing. Related thereto, the covering device can readily enclose the retaining system located exposed outside the patient, so that the oversize can also be maintained in this area.

It has proven to be particularly expedient to configure the tubular film in the shape of a circular cylinder jacket or a truncated cone jacket, i.e. it is designed rectangular or trapezoidal in an axial section plane. Additionally or alternatively, one axial end of the tubular film can be adapted to the shape of an arm or a leg. Using the oversized opening, it can then be readily pulled over any limbs, to which an external fixator can be applied. In the area of the oversized opening, the covering device can subsequently be attached and sealed to the skin using suitable adhesive strips. In this respect, reference is made to the disclosure content of EP 2 636 417 A1 (see above).

The tubular film of a wound care device according to the invention may be associated with an application aid, such as a stable ring having a circumference adapted to the opening circumference length or a netting. In particular when using an application aid in the form of a stable ring, the desired opening circumference length can also be achieved by stretching a stretchable material, such as a parafilm, used for producing the tubular film.

A wound care device according to the invention can be applied in a particularly variable manner in caring for wounds in the area of various limbs, which are cared for using an external fixator, if a tubular film, as the case may be, of low extensibility, has, over an axial length of 40 cm or more, particularly 50 cm or more, particularly preferred 75 cm or more, a circumferential length, i.e. a length in a plane perpendicular to the axis, of 70 cm or more, in particular 100 cm or more.

Within the framework of the invention, it has also proven to be expedient if, in its application, the tubular film of a wound care device according to the invention is customized in accordance with the anatomical circumstances. Related thereto, it is expedient if the tubular film is provided with visible markings, such as length markings facilitating adaptation to the wound space and indicating the axial length of corresponding tubular film sections.

The “pin-track infection” frequently observed in treatments using an external fixator, i.e. an infection of the soft tissue around the pin locations, i.e. the points, at which the retaining system penetrates the skin, can be avoided if the covering device is associated with a filling device for filling a cavity formed during generation of a negative pressure between the covering device and the skin of the patient between the patient's skin, the covering device and a stabilizing device penetrating the patient's skin. In this way, a wound healing-promoting negative pressure can be maintained in the entire wound space, including the pin location.

The filling device can encircle rods of the external fixator penetrating the patient's skin, wherein an outer periphery of the filling device may be designed in the shape of a truncated cone. In this way, the filling device may have, in an axial plane of the filling device, a slit facilitating introduction of the rods of the external fixator into a cavity that axially traverses the filling device.

Alternatively or in addition to filling devices having a truncated cone jacket-shaped periphery, filling devices in the form of deformable gauze may be used.

Expediently, the tubular film of a wound care device according to the invention, as the case may be, together with the filling device, will be enclosed in a sterile or sterilizable package. The wound care tube can then be stored and used in sterile condition. Related thereto, it has proven to be particularly expedient if the tube is enclosed in the package in such a way that removal of the tube from the package does not cause any problems. For this purpose, the tubular film may be enclosed in the sterile package in gathered form or on a support, such as a cardboard carrier, in coiled form. The sterile package may contain additional application aids, such as threads, ears (removal and/or application aid) or similar. Related thereto, it is, for instance, conceivable to provide the tubular film with additional “ears,” by which the tubular film can be grasped and removed without any risk of damage.

To avoid damaging the tubular film during application and/or during the vacuum therapy, it is expedient if the tubular film comprises contact areas of increased tensile strength, as the case may be producible by separate reinforcement strips. These contact areas can come into contact with the stabilizing elements of the external fixator located exposed outside the patient without any risk of damage.

As shown by the preceding explanation of the wound care devices according to the invention, a wound care kit according to the invention has a wound care device according to the invention as well as an external fixator having a stabilizing element, which is located at least partially exposed outside the patient during treatment of a patient, the tubular film of the wound care device being dimensioned in such a way that it can completely enclose the external fixator during treatment of the patient.

If, for instance, an external fixator is used for stabilizing a broken forearm and contains rods, which penetrate the patient's skin and are connected outside the patient using a connecting rod, as the case may be of variable length, the tubular film of a wound care kit according to the invention must be dimensioned in such a way that it can enclose not only the patient's forearm but also the rods penetrating the forearm and the connecting rods in such a way that a negative pressure can be generated within the space formed between the patient's skin and the tubular film and the space containing the external fixator.

The tubular film of a wound care kit according to the invention must be dimensioned in a similar manner if the external fixator is used for fixing a lower leg or thigh fracture, a pelvic fracture, a broken hand or foot or similar. In each case, it will be necessary to assure that the tubular film can enclose not only the body part to be treated but also the corresponding external fixator during treatment.

A wound care kit according to the invention may contain stabilizing elements in the form of nails, pins or similar. To avoid damage of the tubular film during transport and storage of a wound care kit according to the invention, the nails, pins, screws or similar may be associated with covers or padding. For this purpose, one embodiment of the invention considers having the stabilizing device as a whole kept separate from the tubular film using a cover or padding. Alternatively, embodiments are also conceivable, in which each individual stabilizing element, such as each individual screw, each individual nail or similar, is associated with an individual cover or padding. In the event that, despite the measures described, damages to the tubular film occur, in a particularly preferred embodiment of the invention, it is considered for the wound care kit to also have, in addition to the stabilizing elements, the tubular film and, as the case may be, the filling devices, repair means, such as spray plaster, a gel compound or similar for sealing leaks in the tubular film. The filling devices described can also be provided in the wound care kit in the form of a gel compound, such as a wound silicone, or a filling cone.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

The invention will be explained hereinafter with reference to the drawings, to which express reference is made with respect to all the details relevant for the invention and not explained in detail in the specification.

In the drawings:

FIG. 1a-FIG. 1d is a wound care kit according to a first embodiment of the invention,

FIG. 2a-FIG. 2d is a wound care kit according to a second embodiment of the invention,

FIG. 3a-FIG. 3d is a wound care kit according to a third embodiment of the invention,

FIG. 4a-FIG. 4d is a wound care kit according to a fourth embodiment of the invention,

FIG. 5 is a wound care kit according to a fifth embodiment of the invention, and

FIG. 6 is a wound care kit according to a sixth embodiment of the invention.

### DETAILED DESCRIPTION OF PREFERRED EMBODIMENT

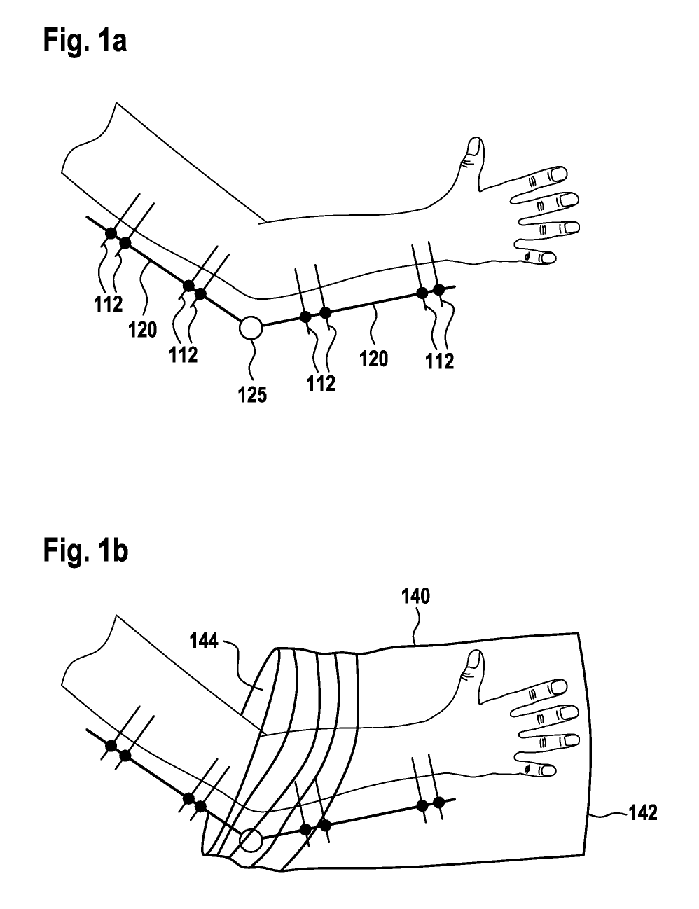

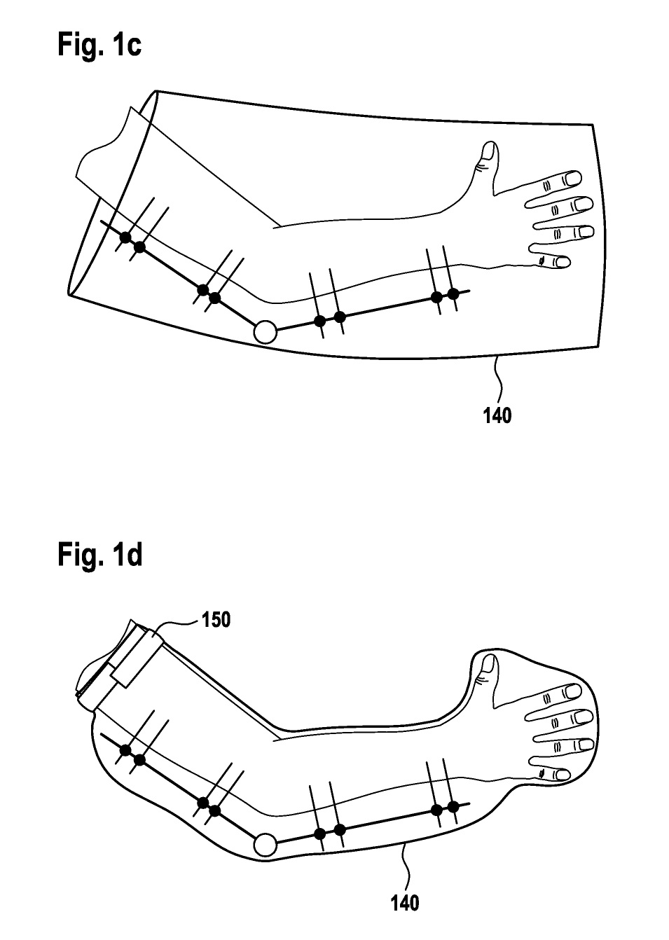

The wound care kit illustrated in FIG. 1 comprises a total of eight stabilizing elements penetrating a patient's skin in the forearm and upper arm areas in the form of nails 112 as well as connecting rods 120, which are interconnected in articulated form across a joint 125. The connecting rods 120 and the joint 125 are located exposed outside the patient and form an external fixator by means of which a fracture in the forearm, upper arm and elbow areas can be fixed. For treating a wound located in the area of the external fixator, a tubular film 140, of a water vapor-permeable material and hermetically sealed on one axial end 142, is pushed over the patient's arm and the external fixator. Opening 144 of the tubular film, facing away from hermetically sealed end 142, is dimensioned in such a way that the introduction of the patient's limbs into the tubular film by the external fixator is not obstructed. The process of introducing the patient's arm into the tubular film is shown in FIGS. 1b and 1c. Once the limb to be treated, including the wound to be treated, is completely enclosed in tubular film 140, the edge of the tubular film encircling opening 144 is hermetically connected to the patient's skin using adhesive films 150 in such a way that, between the patient's skin and the tubular film, a hermetically sealed but water vapor-permeable wound space is created. In this wound space, across a suction port (not illustrated), a negative pressure can be generated, which promotes wound healing.

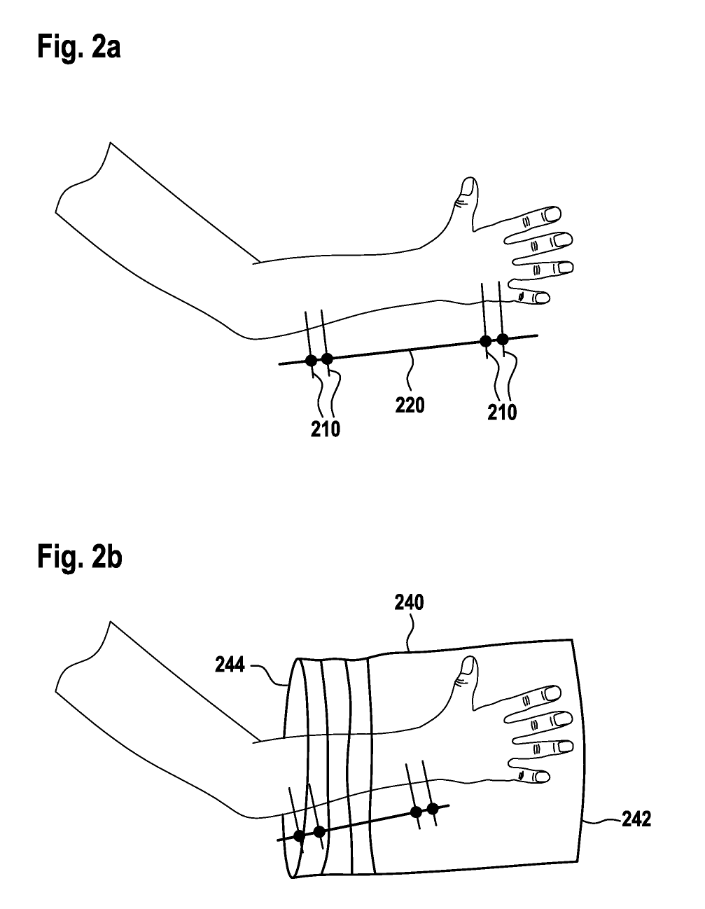

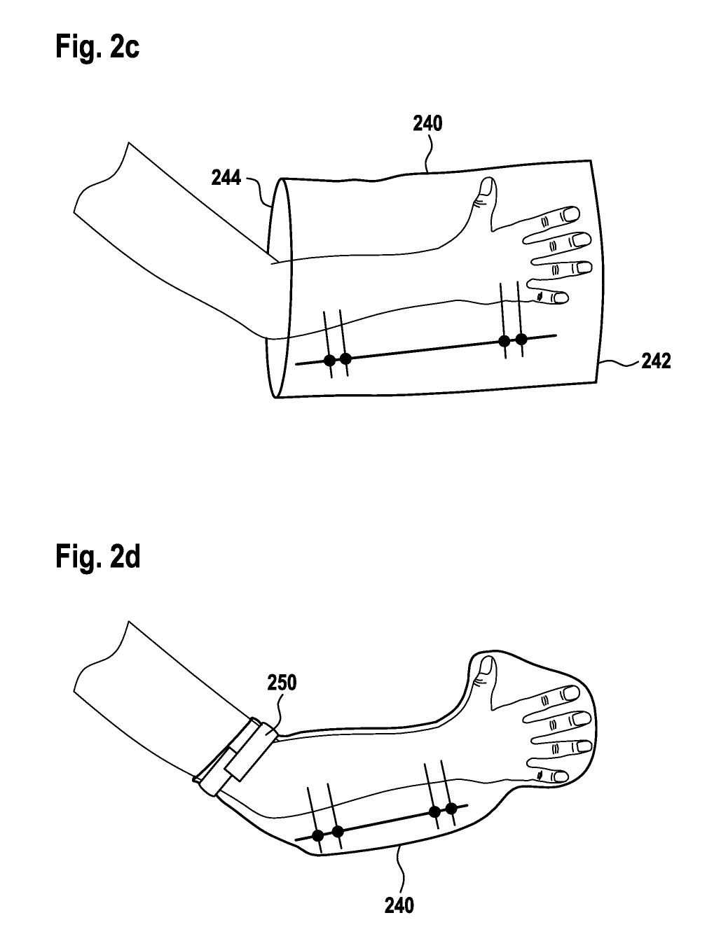

The embodiment of the invention illustrated in FIG. 2 essentially differs from the embodiment illustrated based on FIG. 1 only in that it has an external fixator having only four nails 210 and a connecting rod 220 penetrating the patient's skin. The external fixator is designed for fixing a fracture in the wrist area. Tubular film 240 of the embodiment explained based on FIG. 2 is designed shorter than tubular film 140 of the embodiment explained based on FIG. 1 because it needs to reach only as far as the elbow area. There, tubular film 240, which is hermetically sealed at an axial end 242, is hermetically connected to the patient's skin using adhesive films 250. In this way, a wound space is created, which is hermetically sealed but water vapor-permeable and can be evacuated across a suction port (not illustrated).

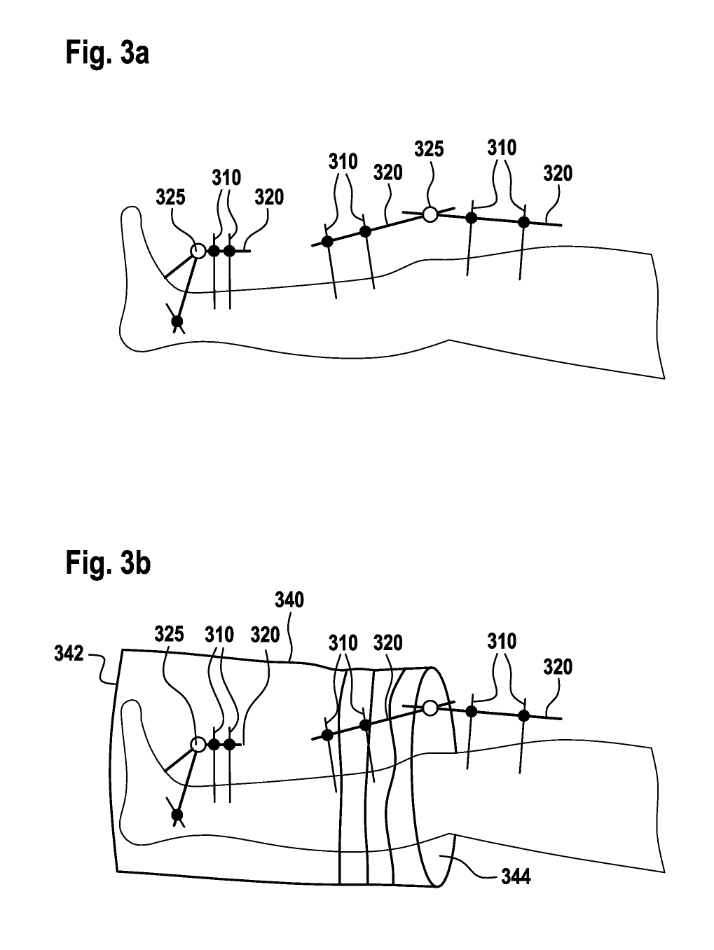

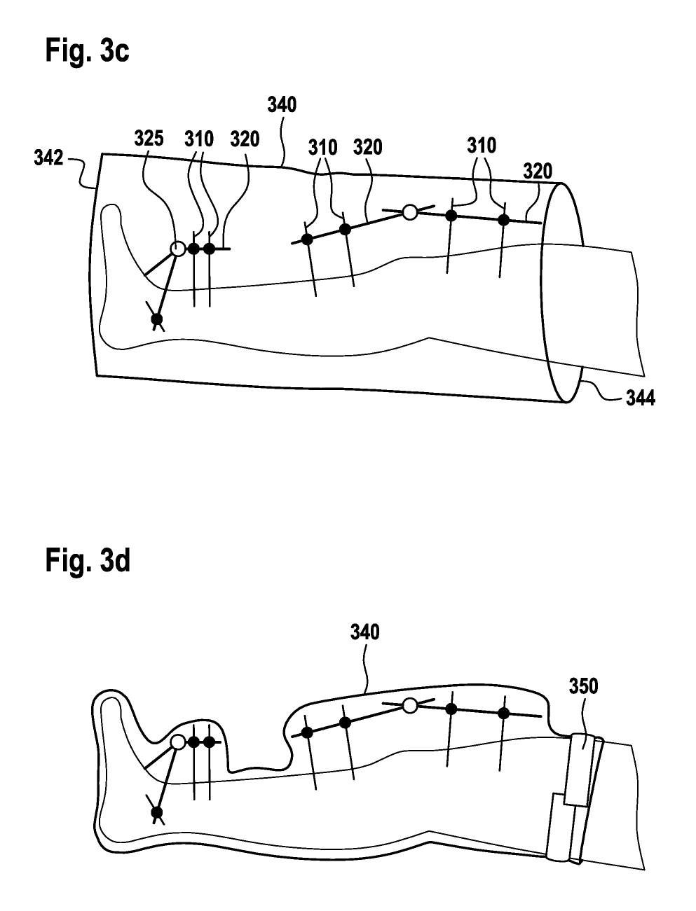

The embodiment shown in FIG. 3 essentially differs from the embodiment illustrated based on FIGS. 1 and 2 in that it is designed for treating a wound in the area of a leg. Related thereto, the embodiment of the invention illustrated in FIG. 3 comprises a total of six nails 310 and three connecting rods 320 penetrating the patient's skin. Two of the nails penetrating the patient's skin are arranged in the area of the patient's ankle, an additional stabilizing aid 325 being provided to bridge the ankle. The remaining nails 310 are arranged on both sides of the patient's knee joint and interconnected across connecting rods 320, which are interconnected across a joint 325 in the area of the knee joint. Similar to the embodiments of the invention illustrated based on FIGS. 1 and 2, the wound care kit shown in FIG. 3 comprises, hermetically sealed at an axial end 342, a tubular film 340, which is dimensioned in such a way that one of its openings 344 can be pushed over the leg and the external fixator as shown in FIGS. 3b and 3c. Moreover, an edge encircling opening 344 can be hermetically connected to the patient's skin using adhesive films 350, thereby creating a hermetic, but water vapor-permeable wound space, which can be evacuated across a suction port (not illustrated). In the embodiment of the invention illustrated based on FIG. 3, the axial length of the tubular film is more than 90 cm. The opening circumference length in the area of opening 344 of the tubular film is more than 120 cm.

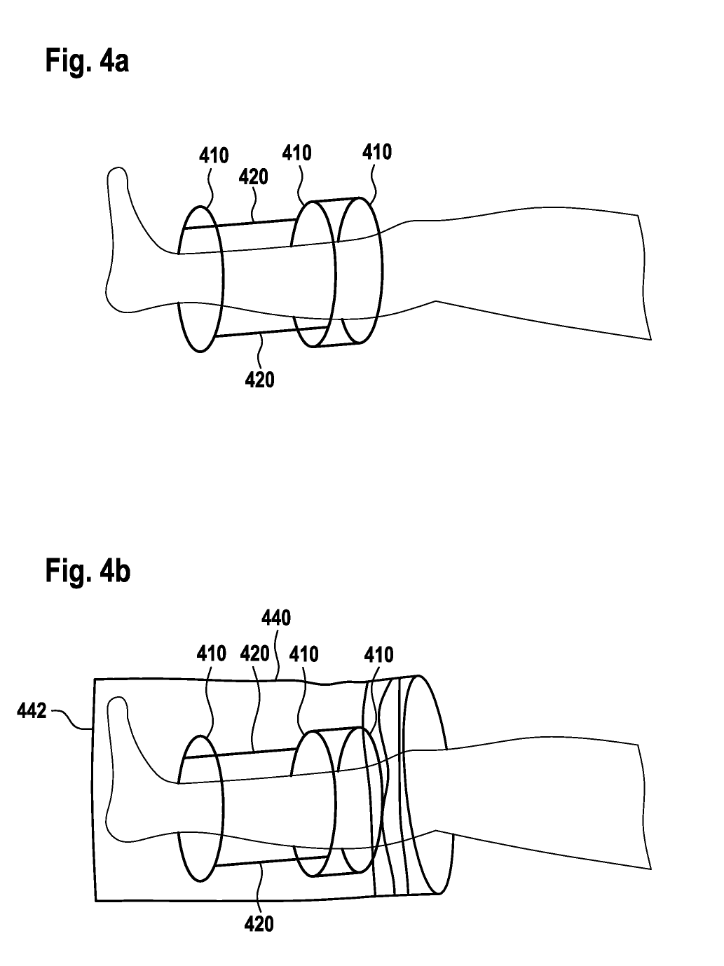

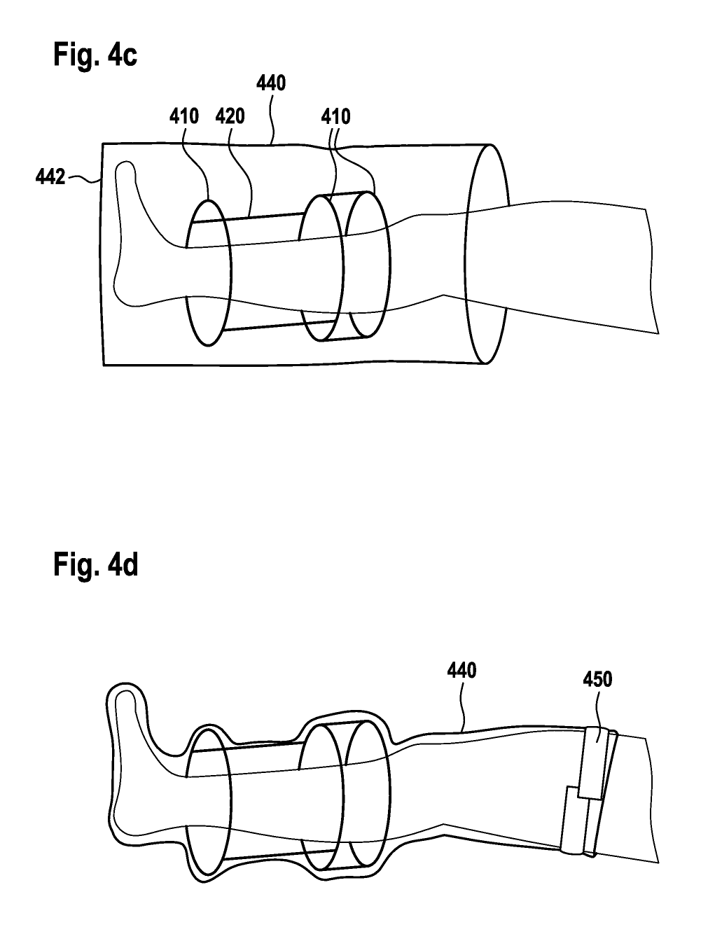

The embodiment of the invention illustrated in FIG. 4 essentially differs from the embodiment shown based on FIG. 3 only in that it is intended for treating a wound in the area of a lower leg fracture. The external fixator of the embodiment of the invention shown in FIG. 4 comprises three stabilizing rings 410 encircling the patient's lower leg which, using nails penetrating the patient's skin, are attached to the bone fragments to be interconnected. The encircling rings 410 are interconnected using connecting rods 420. For creating a hermetically sealed wound space in the area of the lower leg, a tubular film 400, hermetically sealed at an axial end 442, is pushed over the lower leg having the external fixator attached to it and hermetically connected to the patient's skin using adhesive films 450. In doing so, the hermetic connection to the patient's skin according to FIG. 4d can take place above the patient's knee joint. As in the other embodiments of the invention, the wound space can be evacuated using a suction port, not shown in the drawing, in order to promote wound healing in this manner.

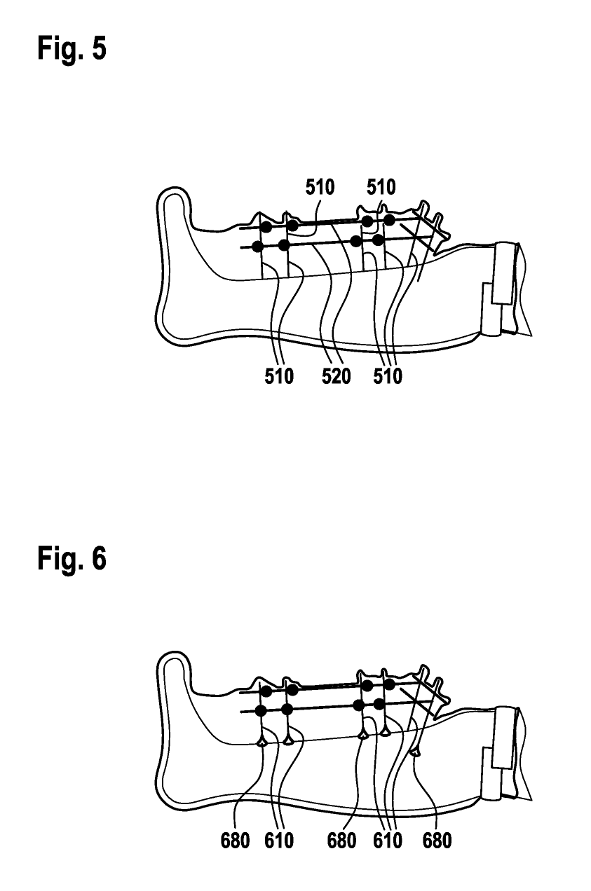

The embodiment of the invention illustrated in FIG. 5 essentially differs from the embodiment illustrated based on FIG. 4 only in that the nails penetrating the patient's skin are interconnected using connecting rods 520, without stabilizing rings being used. According to FIG. 6, in the area of transition between the nails 610 and the patient's skin, truncated cone jacket-shaped filling devices 680 may be provided, by means of which the formation of a cavity between the nails, the tubular film and the patient's skin can be prevented.

The invention is not limited to the embodiments explained based on the drawing. Instead, with appropriate dimensioning of the tubular film, the invention can also be used for vacuum treatment in the pelvic area, the upper arm area, the thigh area, etc.

While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

1-13. (canceled)

14. A wound care device comprising:
a tubular film having an opening circumference length of 80 cm or more and at least portions of the film being water vapor-permeable, wherein the tubular film is used to surround a wound to produce an enclosed wound space in which negative pressure can be generated.

15. The wound care device of claim 14, further comprising a suction port across which a negative pressure can be generated in the wound space.

16. The wound care device of claim 14, wherein one end of the tubular film is sealed.

17. The wound care device of claim 14, wherein the tubular film is a circular cylinder shape.

18. The wound care device according to claim 14, further comprising an application aid.

19. The wound care device of claim 18, wherein the application aid is a ring adapted to the opening circumference length of the tubular film.

20. The wound care device of claim 14, wherein the tubular film has an axial length of 40 cm or more and a circumference length of 100 cm or more.

21. The wound care device of claim 14, further comprising a visible marking indicating axial length on the tubular film.

22. The wound care device of claim 14 wherein the tubular film surrounds a wound in the area of an external fixator, comprising at least one nail penetrating the skin of the patient, to produce a wound space, and the wound care device further comprises at least one filling device, wherein the filling device fills a cavity space between the skin of the patient, the nail, and the tubular film during generation of a negative pressure in the wound space.

23. The wound care device of claim 22, wherein the filling device is a truncated cone shape and defines a slit facilitating introduction of the nail into the filling device.

24. The wound care device of claim 14, wherein the tubular film further comprises contact areas of increased tensile strength.

25. A wound care system comprising:
a wound care device according to claim 14;
an external fixator at least partially exposed outside of a patient during treatment; and
wherein the tubular film is dimensioned to fully enclose the external fixator during treatment of a patient.

26. The wound care system of claim 25, further comprising means for repairing the tubular film.

27. The wound care system of claim 25, further comprising at least one filling device.

28. The wound care system of claim 27, wherein the external fixator comprises a nail penetrating the patient's skin and the filling device comprises a truncated cone shape that defines a slit facilitating introduction of the nail into the filling device.

29. A method of treating a wound in the area of an external fixator immobilizing a patient body part using vacuum therapy, the method comprising:
pulling a water vapor-permeable tubular film having an oversized opening of a circumference length of 80 cm or more over an external fixator comprising a portion penetrating the skin of the patient;
sealing the tubular film near the oversized opening to the skin of the patient to create a hermetically sealed and water vapor-permeable wound space around the external fixator; and generating a negative pressure in the wound space to promote wound healing.

30. The method of claim 29 further comprising encircling the portion of the external fixator penetrating the skin with a filling device, wherein the cavity formed during generation of the negative pressure in the wound space between the portion of the external fixator penetrating the skin, the skin of the patient, and the tubular film is filled.

31. The method of claim 29 further comprising using an application aid to facilitate pulling the tubular film over the external fixator.

32. A covering device for a wound in the area of an external fixator immobilizing a patient body part, the covering device comprising:
a water vapor-permeable tubular film having an oversized opening of a circumference length of 80 cm or more and a sealed end;
at least one reinforcement strip that increases the tensile strength of the tubular film in at least one contact area; and
a suction port, wherein when the tubular film is pulled over a wound in the area of an external fixator and sealed to create a wound space, a negative pressure can be generated across the suction port.

33. The covering device of claim 32, further comprising an application aid selected from the group consisting of a stable ring, threads, and ears.
